(19) United States
(12) Patent Application Publication
(10) Pub. No.: US 2013/0274611 A1
(43) Pub. Date: Oct. 17, 2013

(54) OPTICAL INTERFACE SYSTEMS FOR APPLICATION OF OPTICAL SIGNALS INTO TISSUE OF A PATIENT

(71) Applicants: Paulo E. X. Silveira, Boulder, CO (US); Sarah Hayman, Boulder, CO (US)

(72) Inventors: Paulo E. X. Silveira, Boulder, CO (US); Sarah Hayman, Boulder, CO (US)

(73) Assignee: NELLCOR PURITAN BENNETT LLC, Boulder, CO (US)

(21) Appl. No.: 13/448,544

(22) Filed: Apr. 17, 2012

(51) Int. Cl. A61B 5/00; A61B 6/00

(52) U.S. Cl. 600/476

(57) ABSTRACT

Systems and methods for applying optical signals onto tissue of a patient are provided herein. In one example, a system to optically analyze tissue of a patient is provided. The system includes a measurement system configured to transfer source optical signals over an optical link, receive measurement optical signals over the optical link, and identify a value of a physiological parameter of the patient. The optical link comprises a first source portion with a first link property configured to carry the source optical signals and a second source portion with a second link property coupled to the first source portion and configured to carry the source optical signals. The system includes a tissue interface assembly configured to receive the source optical signals transferred over the optical link, emit the source optical signals into the tissue, and receive the measurement optical signals from the tissue for transfer over the optical link.

### TECHNICAL FIELD

Aspects of the disclosure are related to the field of medical devices, and in particular, optical interface systems for application of optical signals into tissue of a patient and optical measurement of physiological parameters of blood and tissue.

### TECHNICAL BACKGROUND

Various devices, such as pulse oximetry devices or photon density wave (PDW) devices, can measure parameters of blood or tissue in a patient, such as heart rate and oxygen saturation of hemoglobin, among other parameters. These devices are non-invasive measurement devices, typically employing solid-state lighting elements, such as light-emitting diodes (LEDs) or solid state lasers, to introduce light into the tissue of a patient. The light is then detected and analyzed to determine the parameters of the blood and blood flow in the patient.

In many measurement devices, the measurement and processing systems are located remotely from various optical elements used for interfacing optical signals with the tissue of the patient. This configuration can provide some patient mobility by using a flexible fiber optic cable between the equipment. However, having a long cable can introduce errors and attenuation into the measurement and subsequent processing of the optical signals due in part to length-dependent limitations of the long optical cables. Likewise, flexible optical fibers can have poorer optical transmission properties than more rigid optical fibers, making interfacing with tissue of a patient awkward while simultaneously maintaining high optical signal integrity.

### OVERVIEW

Systems and methods for applying optical signals onto tissue of a patient are provided herein. In one example, a system to optically analyze tissue of a patient is provided. The system includes a measurement system configured to transfer a plurality of source optical signals over an optical link, receive a plurality of measurement optical signals over the optical link, and process at least the plurality of measurement optical signals to identify a value of a physiological parameter of the patient. The optical link includes a first source portion with a first link property configured to carry the plurality of source optical signals and a second source portion with a second link property coupled to the first source portion and configured to carry the plurality of source optical signals. The system also includes a tissue interface assembly configured to receive the plurality of source optical signals transferred over the optical link, emit the plurality of source optical signals into the tissue, and receive the plurality of measurement optical signals from the tissue for transfer over the optical link.

In another example, a method of operating a system to optically analyze tissue of a patient is provided. The method includes, in a measurement system, transferring a plurality of source optical signals over an optical link, receiving a plurality of measurement optical signals over the optical link, and processing at least the plurality of measurement optical signals to identify a value of a physiological parameter of the patient. The method also includes, in a first source portion of the optical link, carrying the plurality of source optical signals using a first link property, and in a second source portion of the optical link coupled to the first source portion, carrying the plurality of source optical signals using a second link property. The method also includes, in a tissue interface assembly, receiving the plurality of source optical signals transferred over the optical link, emitting the plurality of source optical signals into the tissue, and receiving the plurality of measurement optical signals from the tissue for transfer over the optical link.

In another example, a system to optically analyze tissue of a patient is provided. The system includes a measurement system configured to transfer a plurality of source optical signals over an optical link, receive a plurality of measurement optical signals over the optical link, and process at least the plurality of measurement optical signals to identify a value of a physiological parameter of the patient. The optical link includes a plurality of source optical fibers each coupled at first ends to associated optical sources of the measurement system that generate individual ones of the plurality of source optical signals, and the plurality of source optical fibers further coupled to an intermediate optical fiber at second ends, where the intermediate optical fiber is configured to carry the plurality of source optical signals for delivery to a tissue interface assembly. The optical link further includes a plurality of measurement optical fibers configured to receive the plurality of measurement optical signals transferred by the tissue interface assembly at third ends, and each of the plurality of measurement optical fibers further coupled to associated optical detectors of the measurement system at fourth ends. The tissue interface assembly includes a tissue interface pad configured to interface with the tissue to emit the plurality of source optical signals into the tissue and receive the plurality of measurement optical signals from the tissue. The tissue interface assembly also includes a tissue interface optical link coupled to the intermediate optical fiber and to the plurality of measurement optical fibers.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 is a system diagram illustrating a system for applying optical signals to tissue of a patient.

FIG. 2 is a flow diagram illustrating a method of operation of a system for applying optical signals to tissue of a patient.

FIG. 3 is a system diagram illustrating a system for applying optical signals to tissue of a patient.

FIG. 4 is a system diagram illustrating a system for applying optical signals to tissue of a patient.

FIG. 5 is a system diagram illustrating a system for applying optical signals to tissue of a patient.

### DETAILED DESCRIPTION

Various physiological parameters of tissue and blood of a patient can be determined non-invasively, such as optically. In one example, optical signals introduced into the tissue of the patient are modulated according to a high-frequency modulation signal to create a photon density wave (PDW) optical signal in the tissue undergoing measurement. Due to the interaction between the tissue or blood and the PDW optical signal, various characteristics of the PDW optical signal can be affected, such as through scattering or propagation by various components of the tissue and blood. The various physiological parameters can include any parameter associated with the blood or tissue of the patient, such as hemoglobin concentration (tHb), regional oxygen saturation (rSO2), arterial oxygen saturation (SpO2), heart rate, lipid concentrations, among other parameters, including combinations thereof.

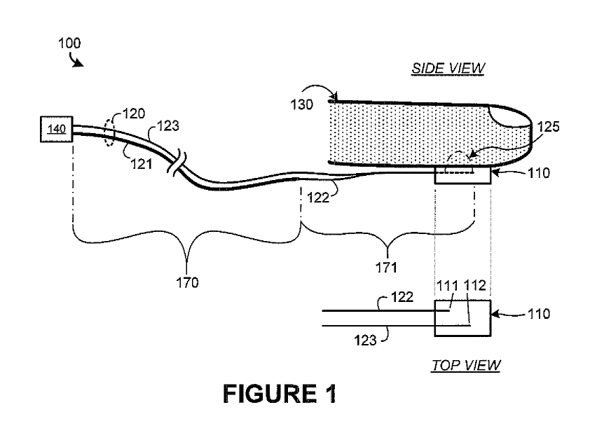

As a first example of a system for applying optical signals to tissue of a patient, FIG. 1 is presented. FIG. 1 includes system 100, which further includes tissue interface pad 110, optical link 120, tissue 130, and measurement system 140. A top view and side view of some elements of system 100 are included in FIG. 1 to highlight these particular elements. It should be understood the features of FIG. 1 are merely intended to highlight various elements of system 100, and are not intended to be exact wireframe representations of the elements of system 100; variations are possible.

In operation, optical signals generated by measurement system 140 are applied to tissue 130 for measurement of a physiological parameter, as indicated by optical signals 125. In this example, optical signals are transferred via optical link 120 to tissue interface pad 110 for application into tissue 130. Optical signals 125 are detected through tissue 130 and transferred to measurement system 140 over optical link 120. Optical link 120 includes two optical pathways in this example, a first pathway for source optical signals which includes optical fibers 121-122, and a second pathway for measurement optical signals which includes optical fiber 123. Optical fiber 122 is terminated at location 111 of tissue interface pad 110 and optical fiber 123 is terminated at location 112 of tissue interface pad 110.

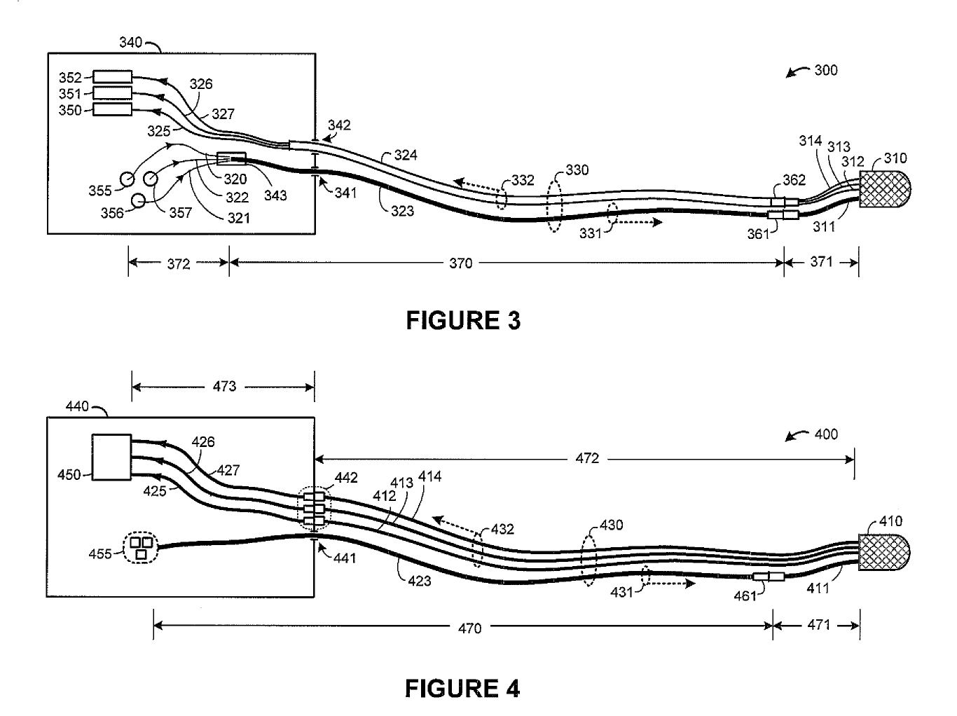

Also, FIG. 1 shows two portions of optical link 120, namely first portion 170 and second portion 171. In this example, these portions 170-171 are associated with the first optical pathway for source optical signals. First source optical fiber 121 is included in first portion 170 of optical link 120, and first portion 170 has a first link property. Likewise, second source optical fiber 122 is included in second portion 171 of optical link 120, and second portion 171 has a second link property. Optical fiber 121 and optical fiber 122 are coupled together for transfer of optical signals. Although the second optical pathway for measurement optical signals is shown in FIG. 1 with a single portion including optical fiber 123, in further examples, the second optical pathway for measurement optical signals also has at least two portions which can include separate optical fibers with different link properties. Further examples are discussed below in FIGS. 3-5.

Advantageously, optical signals carried over an optical pathway can traverse multiple different sequential portions such as different optical fiber portions. These multiple portions can each have different optical or physical properties. Thus, in one example, flexible and less expensive (but more attenuating) optical fiber can be employed for a short length in disposable or replaceable components as well as to ease interfacing with patient tissue, while rigid and more expensive (but less attenuating) optical fiber can be employed for a longer length in non-disposable components and can allow for less signal degradation overall than a single long portion of flexible and less expensive (but more attenuating) optical fiber.

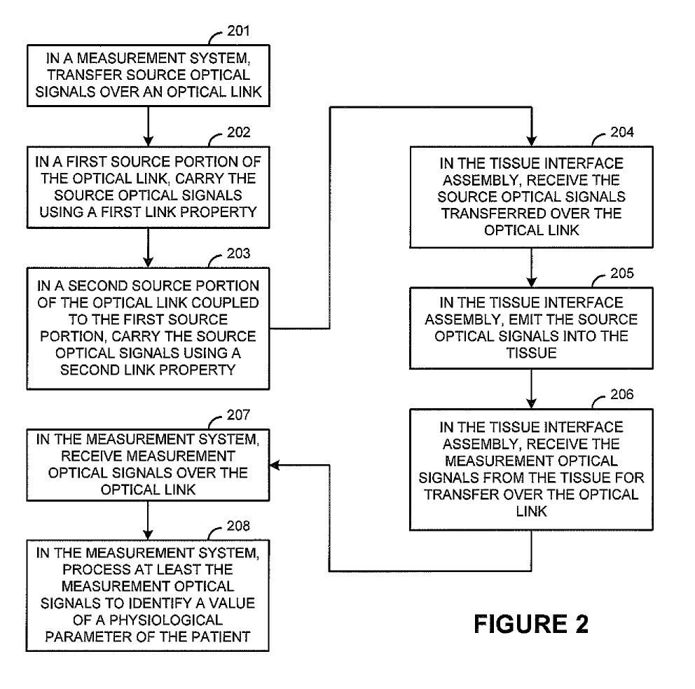

FIG. 2 is a flow diagram illustrating a method of operation of system 100 for applying optical signals to tissue of a patient. The operations of FIG. 2 are referenced herein parenthetically. In FIG. 2, measurement system 140 transfers (201) source optical signals over optical link 120. A first source portion of optical link 120, which includes optical fiber 121, carries (202) the source optical signals using a first link property. A second source portion of optical link 120 coupled to the first source portion, namely optical fiber 122, carries (203) the source optical signals using a second link property.

The first link property and the second link property can be one or more among various link properties, such as an optical attenuation property, a minimum bend radius, a link length, an optical fiber material or composition, numerical aperture, or a cross-sectional thickness. The optical attenuation property can include a loss factor, measured in decibels (dB) per unit length, for an optical signal carried by the associated optical fiber. The numerical aperture (NA) of an optical fiber corresponds to a modal dispersion property which can affect a bandwidth or phase dispersion quality of an optical link. Thus, an optical fiber with a lower NA carries fewer modes than a fiber with a higher NA, resulting in a reduction in the total amount of modal dispersion.

In some examples, optical fiber 121 is of a first length and composed of a first material having a first attenuation level per unit length, while optical fiber 122 is of a second length and composed of a second material having a second attenuation level per unit length. The various materials can include glasses (such as SiO₂ or silica glass), polymethyl methacrylate (PMMA), plastics, or other optically transmissive materials, including associated cladding material. Typically, the optical fibers are selected to be transmissive in a wavelength range from 630 nanometers (nm) to 1300 nm, in part due to the corresponding range over which biological tissue presents low light absorption. Optical fiber 121 can be coupled to optical fiber 122 using a butt coupling technique. The butt coupling technique can include cutting and polishing the ends of each optical fiber and mating using an optically transmissive adhesive. In other examples, an optical connector is employed to couple optical fiber 121 and optical fiber 122, which may include non-physical contact (NPC) type connectors or physical contact (PC) type connectors.

Tissue interface assembly 110 receives (204) the source optical signals transferred over optical link 120, emits (205) the source optical signals into tissue 130, and receives (206) the measurement optical signals from tissue 130 for transfer over optical link 120. Tissue interface pad 110 couples to biological tissue, namely tissue 130, to allow for introduction of optical signals received over optical link 120 into tissue 130. Tissue interface pad 110 also allows for receipt of optical signals propagated through tissue 130 into optical link 120.

Measurement system 140 receives (207) the measurement optical signals over optical link 120, and processes (208) at least the measurement optical signals to identify a value of a physiological parameter of the patient. Upon receiving optical signals over optical link 120 after propagation through tissue 130, measurement system 140 may process detected optical signals to determine various characteristics of the detected optical signals. Physiological parameters of the tissue and patient can then be identified based on the various characteristics of the detected optical signals.

Referring back to FIG. 1, tissue interface pad 110 comprises a physical structure having a surface that couples to biological tissue, namely tissue 130. The surface includes at least one optical signal emission point 111 and may include at least one optical signal detection point 112. Tissue interface pad 110 includes a mechanical arrangement to position and hold optical fibers 122-123 in a generally parallel arrangement to tissue 130. Other optical fiber arrangements can be employed, such as non-parallel to tissue 130. These mechanical arrangements can include grooves, channels, holes, snap-fit features, or other elements to route optical fibers 122-123 to a desired position in tissue interface pad 110. Tissue interface pad 110 may be comprised of plastic, foam, rubber, glass, metal, adhesive, or some other material, including combinations thereof. Typically, the materials selected for tissue interface pad 110 include biocompatible materials. Specifically, biocompatible materials are inert, non-toxic and hypoallergenic, and typically exclude latex and heavy metals, for example. Also, the materials selected for tissue interface pad 110 are typically selected to avoid materials that are difficult to clean. In some examples, tissue interface pad 110 is comprised of optically transmissive materials, such as optically transmissive plastic, glass, acrylic glass, PMMA, or other materials, including combinations thereof. Optically transmissive adhesives can also be employed in tissue interface pad 110, such as to mate optical fibers 122-123 to optical interface elements of tissue interface pad 110. These optical adhesives can comprise compositions which are cured using ultraviolet (UV) light. Other optically transmissive adhesives can be employed, including combinations thereof. Various optical interfacing elements can be employed to optically couple optical signals carried by optical fibers 122-123 to tissue 130, such as prisms, reflective surfaces, refractive materials, or the like.

Tissue 130 is shown in FIG. 1 as a finger of a patient. It should be understood that tissue 130 can be any tissue portion of a patient, such as a finger, toe, arm, leg, earlobe, torso, forehead, or other tissue portion of a patient. In this example, tissue 130 is a portion of the tissue of a patient undergoing measurement of a physiological blood parameter. The wavelength of signals applied to the tissue can be selected based on many factors, such as optimized to a wavelength strongly absorbed by hemoglobin, lipids, proteins, or other tissue and blood components of tissue 130.

Measurement system 140 includes optical interfaces, digital processors, computer systems, microprocessors, circuitry, non-transient computer-readable media, user interfaces, or other processing devices or software systems, and may be distributed among multiple processing devices. Measurement system 140 may also include photon density wave (PDW) generation and measurement equipment, electrical to optical conversion circuitry and equipment, optical modulation equipment, and optical waveguide interface equipment. Measurement system 140 also includes optical emitter elements such as lasers, laser diodes, solid-state lasers, light-emitting diodes (LEDs), or other optical emitter devices, along with associated driving circuitry. Measurement system 140 also includes optical detector elements, such as a photodiode, phototransistor, avalanche photodiode (APD), photomultiplier tube, charge coupled device (CCD), or other optoelectronic sensor, along with associated receiver circuitry such as amplifiers or filters. Optical couplers, adhesives, cabling, or attachments can be included to optically mate emitter or detector elements to optical fibers 121 and 123.

Optical fibers 121-123 each comprise an optical waveguide, and each use glass, polymer, air, space, or some other material as the transport media for transmission of light, and can each include multimode fiber (MMR) or single mode fiber (SMF) materials. A sheath or loom can be employed to bundle optical fibers 121-123 together or with further optical links for convenience, as indicated by optical link 120. One end of each of optical fibers 121 and 123 mates with an associated optical driver or detector component of measurement system 140, and an end of each of optical fibers 122-123 is configured to terminate in tissue interface pad 110 for optically interfacing with tissue 130. Each of optical fibers 121-123 may include many different signals sharing the same associated link, as represented by the associated lines in FIG. 1, comprising channels, forward links, reverse links, frequencies, wavelengths, modulation frequencies, carriers, timeslots, spreading codes, logical transportation links, or communication directions.

Also, although FIG. 1 illustrates optical fibers 121-123, it should be understood that any number of input links and measurement links can be included, as well as any associated optical source and detector equipment. For example, tissue interface pad 110 may route many optical fibers to different physical locations on tissue 130, and these optical fibers can carry optical signals of different wavelengths. Alternatively, or in addition, tissue interface pad 110 may have measurement links positioned at different distances from input links or positioned over different anatomical structures. Also, although the optical source of FIG. 1 is shown as optical fibers 121-122 in this example, in further examples a direct light source can be included in tissue interface 110 and applied to tissue 130. Such direct light sources can include light-emitting diodes (LEDs), laser sources, or other signal sources, including combinations thereof.

The term ‘optical’ or ‘light’ is used herein for convenience. It should be understood that the applied and detected signals are not limited to visible light, and can comprise any photonic, electromagnetic, or energy signals, such as visible, infrared, near-infrared, ultraviolet, radio, x-ray, gamma, or other signals. Additionally, the use of optical fibers or optical cables herein is merely representative of a waveguide used for propagating signals between a transceiver and tissue of a patient. Suitable waveguides would be employed for different electromagnetic signal types.

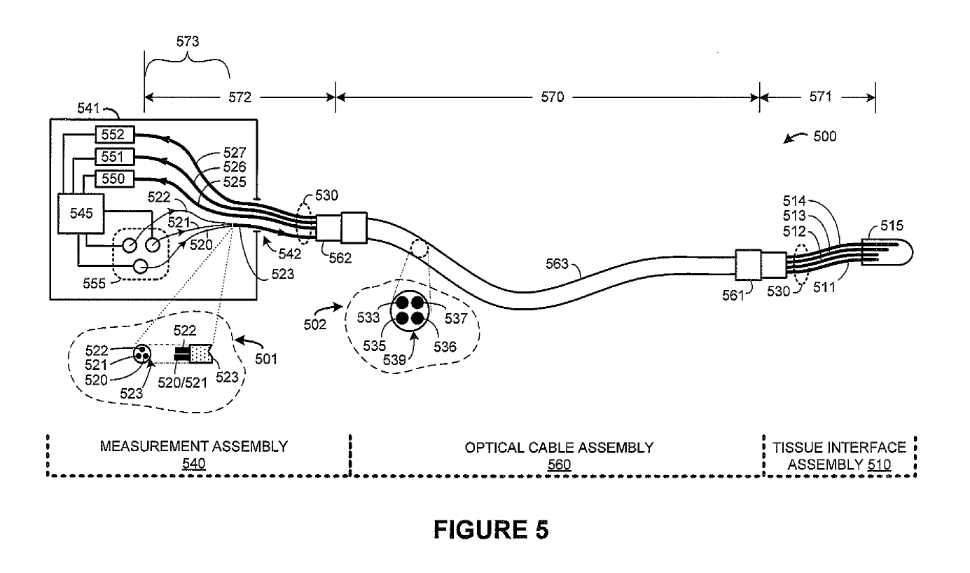

FIGS. 3-5 each detail further examples of systems for applying optical signals to tissue of a patient. FIG. 3 illustrates a first configuration for coupling a tissue interface pad to a measurement system. FIG. 4 illustrates a second configuration for coupling a tissue interface pad to a measurement system. FIG. 5 illustrates a third configuration for coupling a tissue interface pad to a measurement system, with an optical cable portion which can be separately disconnected.

FIG. 3 is a system diagram illustrating system 300 for applying optical signals to tissue of a patient. System 300 includes tissue interface pad 310, optical link 330, and measurement system 340. In operation, optical signals generated by optical sources 355-357 of measurement system 340 are applied to tissue of a patient for measurement of a physiological parameter. Tissue of a patient is not included in this example for clarity, but can be coupled to tissue interface pad 310 as detailed in FIG. 1. Optical signals are transferred via optical link 330 to tissue interface pad 310 for application into the tissue. Optical signals are received through the tissue and transferred to detectors 350-352 of measurement system 340 via optical link 330.

FIG. 3 illustrates portions 370-372 of system 300. These portions are included to highlight different properties of the associated optical fibers of optical link 330. In this example, portion 371 includes optical fibers associated with tissue interface pad 310, which can be a separate assembly than portion 370 associated with measurement system 340. Tissue interface pad 310 and the associated optical fibers 311-314 can be detached via optical connectors 361-362 from the elements of measurement system 340 and associated optical links 323-324. Thus, tissue interface pad 310 can be more readily replaced, disposed, or swapped during subsequent measurements of similar or different patients, or to coupled different types or styles of tissue interface elements to measurement system 340.

Portions 370-372 can include optical fibers of different optical or physical properties, as well as lengths. The optical or physical properties can include optical attenuation properties, minimum bend radii, optical fiber materials or compositions, numerical apertures, or cross-sectional thicknesses, including variations and combinations thereof. The optical attenuation properties can include loss factors, measured in dB per unit length, for an optical signal carried by the associated optical fiber. Portion 370 indicates a first optical fiber length and portion 371 indicates a second optical fiber length in this example, where portion 370 is typically significantly longer than portion 371. Although other lengths can be used, typical lengths are 2.5 meters (m) for portion 370 and 0.5 meters for portion 371. The sum of the lengths of portions 370 and 371 typically are 3 m for use in an operating room. Portion 372 illustrates a sub portion of optical link 330 composed of source optical fibers 320-322. It should be noted the optical links of FIG. 3 are not necessarily drawn to scale.

Thus, optical link 330 includes two optical pathways in this example. A first pathway, namely source pathway 331, is for introduction of source optical signals from measurement system 340 into tissue, and a second pathway, namely measurement pathway 332, is for receipt of optical signals from tissue and into measurement system 340. Source optical fiber 311 and measurement optical fibers 312-314 are each terminated at one end within tissue interface pad 310 for coupling optical signals to and from tissue of a patient.

Source pathway 331 for source or input optical signals includes optical fibers 320-322, optical fiber 323, and optical fiber 311. Optical sources 355-357 are optically coupled to first ends of associated ones of optical fibers 320-322, and are each configured to emit an optical signal of an associated carrier wavelength. The three different optical signals in this example include respective carrier wavelengths of 670 nanometers (nm), 795 nm, and 850 nm, although other wavelengths can be used. The optical coupling between optical sources 355-357 and first ends of associated ones of optical fibers 320-322 can include optically transmissive adhesive, optical connectors, or other optical coupling elements. Second ends of optical fibers 320-322 are coupled to a first end of optical fiber 323. In this example, second ends of all three of optical fibers 320-322 couple without connectors or other coupling equipment to the first end of optical fiber 323 via a butt coupling or butt joint. Ends of each fiber can be butt joined as shown using an optically transmissive adhesive. A rigid strain relief can be placed surrounding the butt joint. The rigid strain relief can be a cylinder made of plastic or metal and glued to fibers very near the joint, as exemplified by strain relief 343 in FIG. 3. Thus, while each of optical fibers 320-322 carries an individual optical signal of a single carrier wavelength, these three individual optical signals are merged into optical fiber 323. Optical fiber 323 then carries all optical signals originally carried by optical fibers 320-322. Optical fiber 323 passes through grommet 341 in a casing of measurement system 340 and terminates at optical connector 361. Grommet 341 could alternatively be an optical connector (NPC or PC) so that the portion of optical fiber 323 which is outside of measurement system 340 can be disconnected. Optical connector 361 couples a second end of optical fiber 323 to a first end of optical fiber 311. Finally, a second end of optical fiber 311 is terminated by tissue interface pad 310 for introduction of optical signals originally generated by optical sources 355-357 into tissue.

Measurement pathway 332 for measurement or output optical signals includes optical fibers 312-314 and optical fibers 325-327. First ends of optical fibers 312-314 are terminated by tissue interface pad 310 for receipt of optical signals from tissue. First ends of optical fibers 312-314 can be terminated at different locations within tissue interface pad 310, such as at different distances from an end of optical fiber 311. Optical fibers 312-314 each receive all optical signals emitted into the tissue, which can include all three optical signals originally generated by optical sources 355-357 and introduced into the tissue. Second ends of optical fibers 312-314 are terminated in optical connector 362. Optical connector 362 couples second ends of optical fiber 312-314 to associated first ends of optical fiber 325-327. Optical connector 362 optically couples optical fiber 312 to optical fiber 325, optical fiber 313 to optical fiber 326, and optical fiber 314 to optical fiber 327. Optical fibers 325-327 are bundled in sheath 324 and passed through grommet 342 of the casing of measurement system 340. Grommet 342 could alternatively be an optical connector (NPC or PC) so that the portion of optical fibers 325-327 which are outside of measurement system 340 can be disconnected. In some examples, the individual optical fibers within sheath 324 are coated or jacketed in an optically absorbing medium or substance, such as polyamide or opaque adhesive, to prevent optical crosstalk between individual optical fibers. Optical fibers 325-327 are then coupled to associated ones of optical detectors 350-352. Sheath 324 can be a co-extruded fiber assembly with a rugged jacket on individual fibers 325-327 which comprises an absorbing medium. Sheath 324 can also include a rugged jacket enclosing all optical fibers 325-327.

In this example, optical fibers 311-314 are composed of first optical fiber material with a first minimum bend radius and a first optical attenuation per unit length. Optical fiber 323 is composed of a second optical fiber material with a second minimum bend radius and a second optical attenuation per unit length. Due in part to its greater length (volume), optical fiber 323 can comprise a higher-quality or less attenuating type of optical fiber, such as an optical fiber composed of glass, and is of a larger minimum bend radius than optical fibers 311-314. Due in part to their smaller length (volume), optical fibers 311-314 can each comprise a lower-quality or higher attenuating type of optical fiber, such as optical fibers composed of PMMA. Thus, optical fiber 323 is generally more rigid, but less attenuating to optical signals than optical fibers 311-314. Optical fibers 320-322 can be of any optical fiber material, but in this example are of a smaller thickness than optical fiber 323. Thus, due to the smaller thicknesses, optical fibers 320-322 can all be mated simultaneously to an end of the larger thickness optical fiber 323. Optical fibers 320-322 can be of a thickness of 200-400 micrometers, and optical fiber 323 can be of a thickness of 800-1000 micrometers, although other thicknesses can be employed. Optical fibers 325-327 can be composed of similar fiber material and thickness as optical fiber 323, although variations are possible. It should be understood that the term thickness used herein refers to a cross-sectional thickness or diameter of the associated optical fiber.

In an alternate example, optical fiber 323 can comprise an optical material, such as glass, with a first minimum bend radius, first attenuation per unit length, and first numerical aperture properties. The optical fiber 323 can be a Fujikura Ltd. S.1000/1100B fiber with a thickness of 1000 micrometers, a minimum bend radius of 220 millimeters, an attenuation of 0.01 dB per meter, and a numerical aperture (NA) of 0.22. Also in this alternate example, optical fiber 311 comprises an optical material with different properties than optical fiber 323, namely polymethyl methacrylate (PMMA). The optical fiber 311 can be an Eska GH 4001 fiber with a thickness of 1000 micrometers, a minimum bend radius of 25 millimeters, an attenuation of 2.5 dB per meter, and a NA of 0.5. Thus, in this example, optical fiber 311 allows for a more flexible and bendable optical link, useful for the last-length portion of optical link 330 which interfaces with a patient. However, optical fiber 311 thus configured would be a more attenuating and lossy optical fiber and not desirable for the entire length of optical link 330. Therefore, optical fiber 323 comprises the main length portion of optical link 330 providing for a less attenuating and lossy optical fiber, albeit with a less flexible/more rigid property. Likewise, in this example, optical fibers 320-322 can each be a Fujikura Ltd. S.200/220B fiber with a thickness of 200 micrometers, a minimum bend radius of 220 millimeters, an attenuation of 0.01 dB per meter, and a NA of 0.22. These optical fibers 320-322 are of a small enough thickness to be simultaneously butt coupled to an end of optical fiber 323. Thicknesses of up to 400 micrometers are possible for butt coupling to optical fiber 323 of 1000 micrometers, although further mating techniques may be needed for 400 micrometer thicknesses than for 200 micrometer thicknesses. Although specific fiber types and properties are discussed above, it should be understood that other fiber types and properties can be employed.

Thus, in the configurations detailed above, a low loss but less flexible fiber is employed for the bulk of the optical link length (i.e. portion 370), while a higher loss but more flexible fiber is employed for portion 371 of the optical link length. Large optical losses over the small portion 371 are therefore limited to the short length of portion 371, while still maintaining flexible optical links for interfacing with tissue of patients.

Returning to the elements of system 300, tissue interface pad 310 may be composed of plastic, foam, rubber, glass, metal, adhesive, or some other material, including combinations thereof. Tissue interface pad 310 includes a generally planar surface configured to interface with tissue to allow for introduction of optical signals into tissue and for receipt of optical signals from tissue. Tissue interface pad 310 also may include elements as discussed above for tissue interface pad 110, although these elements can use different configurations.

Measurement system 340 includes optical sources 355-357 and optical detectors 350-352. Optical sources 355-357 of measurement system 340 can each include solid state lasers, LEDs, vertical-cavity surface-emitting laser (VCSEL), or other optical sources, including combinations thereof. In some examples, optical sources are discrete optical sources, in separate component packages. In other examples, optical sources are packaged into a single component, such as a multiple VCSEL package including all three of optical sources 355-357. Optical detectors 350-352 each comprise an optical detector element, such as a photodiode, phototransistor, avalanche photodiode (APD), photomultiplier tube, charge coupled device (CCD), or other optoelectronic sensor, along with associated receiver circuitry such as amplifiers or filters. Optical detectors 350-352 receive optical signals over associated optical fibers 325-327, and convert the optical signals into corresponding electrical signals. Measurement system 340 can also include digital processors, computer systems, microprocessors, circuitry, non-transient computer-readable media, user interfaces, or other processing devices or software systems, and may be distributed among multiple processing devices. Measurement system 340 can also include photon density wave (PDW) generation and measurement equipment, electrical to optical conversion circuitry and equipment, optical modulation equipment, and optical waveguide interface equipment. Optical couplers, cabling, adhesives, or attachments can be included to optically mate laser or detector elements to optical fibers of optical link 330.

Optical connectors 361-362 may each include non-physical contact (NPC) type connectors or physical contact (PC) type connectors. NPC type connectors employ a gap between the two mating fibers to optically couple signals between the associated optical fibers, whereas PC type connectors employ physical contact of mated optical fibers. A numerical aperture of a first optical fiber coupled in a connector can be of a different numerical aperture of a second optical fiber coupled to the first optical fiber in the connector. Optical connector 362 is shown as a multi-fiber connector, such as a Lightray MPX connector, although other connectors can be employed. In other configurations, optical connectors 361-362 can each include an NPC connector-bulkhead-NPC connector configuration, where NPC connectors are attached to ends of optical fibers to be coupled, and the bulkhead physically couples the NPC connectors to each other.

FIG. 4 is a system diagram illustrating system 400 for applying optical signals to tissue of a patient. System 400 includes tissue interface pad 410, optical link 430, and measurement system 440. In operation, optical signals generated by optical source 455 of measurement system 440 are applied to tissue of a patient for measurement of a physiological parameter. Tissue of a patient is not included in this example for clarity, but can be coupled to tissue interface pad 410 as detailed in FIG. 1. Optical signals are transferred via optical link 430 to tissue interface pad 410 for application into the tissue. Optical signals are received through the tissue and transferred to detector 450 of measurement system 440 via optical link 430.

FIG. 4 illustrates portions 470-473 of system 400. These portions are included to highlight different properties of the associated optical fibers of optical link 430. In this example, portions 471 and 472 include optical fibers associated with tissue interface pad 410, which can be a separate assembly than measurement system 440. Tissue interface pad 410 and the associated optical fibers 411-414 can be detached via optical connectors 461 and 442 from the elements of measurement system 440 and associated optical links 423 and 425-427. Thus, tissue interface pad 410 can be more readily replaced, disposed, or swapped during subsequent measurements of similar or different patients.

Also, portions 470-473 can include optical fibers of different optical or physical properties, as well as lengths. The optical or physical properties can include optical attenuation properties, minimum bend radii, optical fiber materials or compositions, numerical apertures, or cross-sectional thicknesses, including variations and combinations thereof. For the source or input pathway, portion 470 indicates a first optical fiber length and portion 471 indicates a second optical fiber length in this example. Although other lengths can be used, typical lengths are 2.5 meters for portion 470 and 0.5 meters for portion 471, with a total length at approximately 3 m. For the measurement or output pathway, portion 472 indicates a third optical fiber length and portion 473 indicates a fourth optical fiber length in this example. Although other lengths can be used, typical lengths are 3 meters for portion 472 and 0.1 meters for portion 473. It should be noted the optical links of FIG. 4 are not necessarily drawn to scale.

Thus, optical link 430 includes two optical pathways in this example. A first pathway, namely input pathway 431, is for introduction of source optical signals from measurement system 440 into tissue, and a second pathway, namely measurement pathway 432, is for receipt of optical signals from tissue and into measurement system 440. Source optical fiber 411 and measurement optical fibers 412-414 are each terminated at one end within tissue interface pad 410 for coupling optical signals to and from tissue of a patient.

Input pathway 431 for source or input optical signals includes optical fiber 423 and optical fiber 411. Emitter elements of optical source 455 are all optically coupled to a first end of optical fiber 423, and are configured to emit three associated optical signals, each with an associated optical wavelength. The optical coupling between optical source 455 and first end of optical fibers 423 can include optically transmissive adhesive, optical connectors, or other optical coupling elements. Thus, optical source 455 emits three different optical signals, and optical fiber 423 carries all of the individual optical signals. The three different optical signals in this example include respective optical wavelengths of 670 nm, 795 nm, and 850 nm, although other wavelengths can be used. Optical fiber 423 passes through grommet 441 in a casing of measurement system 440 and terminates at optical connector 461. Grommet 441 could alternatively be an optical connector (NPC or PC) so that the portion of optical fiber 423 which is outside of measurement system 440 can be disconnected. Optical connector 461 couples a second end of optical fiber 423 to a first end of optical fiber 411. Finally, a second end of optical fiber 411 is terminated by tissue interface pad 410 for introduction of optical signals originally generated by optical source 455 into tissue.

Measurement pathway 432 for measurement or output optical signals includes optical fibers 412-414 and optical fibers 425-427. First ends of optical fibers 412-414 are terminated by tissue interface pad 410 for receipt of optical signals from tissue. First ends of optical fibers 412-414 can be terminated at different locations within tissue interface pad 410, such as at different distances from optical fiber 411. Optical fibers 412-414 each receive all optical signals emitted into tissue, which can include all three optical signals originally generated by optical source 455 and introduced into the tissue. Second ends of optical fibers 412-414 are terminated in ones of optical connectors 442. Optical connectors 442 couple second ends of optical fibers 412-414 to associated first ends of optical fiber 425-427. Optical connectors 442 pass the optical signals through the casing of measurement system 440, such as through the use of bulkhead-style connectors with associated mating elements. Optical fibers 425-427 are then coupled to optical detector 350.

In this example, optical fibers 411-414 are composed of first optical fiber material with a first minimum bend radius and a first optical attenuation per unit length. Optical fiber 423 is composed of a second optical fiber material with a second minimum bend radius and a second optical attenuation per unit length. Optical fiber 423 can comprise a higher-quality or less attenuating type of optical fiber, such as an optical fiber composed of glass. Optical fibers 411-414 can each comprise a lower-quality or higher attenuating type of optical fiber, such as optical fibers composed of PMMA. Thus, optical fiber 423 is generally less attenuating to optical signals than optical fibers 411-414. Optical fibers 411-414 and 423 can be of a thickness of 1000 micrometers, although other thicknesses can be employed. Optical fibers 425-427 can be composed of similar fiber material and thickness as optical fiber 423, although variations are possible.

Returning to the elements of system 400, tissue interface pad 410 may be composed of plastic, foam, rubber, glass, metal, adhesive, or some other material, including combinations thereof. Tissue interface pad 410 includes a generally planar surface configured to interface with tissue to allow for introduction of optical signals into tissue and for receipt of optical signals from tissue. Tissue interface pad 410 also may include elements as discussed above for tissue interface pad 110 or 310, although these elements can use different configurations.

Measurement system 440 includes optical source 455 and optical detector 450. Optical source 455 can include solid state lasers, LEDs, vertical-cavity surface-emitting laser (VCSEL), or other optical sources, including combinations thereof. In this example, solid state lasers are packaged into a single discrete component, such as a multiple VCSEL package including three solid state laser elements. Optical detector 450 comprises light detector elements, such as a photodiode, phototransistor, avalanche photodiode (APD), photomultiplier tube, charge coupled device (CCD), or other optoelectronic sensor, along with associated receiver circuitry such as amplifiers or filters. Optical detector 450 receives optical signals over optical fibers 425-427, and converts the optical signals into corresponding electrical signals. Measurement system 440 can also include digital processors, computer systems, microprocessors, circuitry, non-transient computer-readable media, user interfaces, or other processing devices or software systems, and may be distributed among multiple processing devices. Measurement system 440 can also include photon density wave (PDW) generation and measurement equipment, electrical to optical conversion circuitry and equipment, optical modulation equipment, and optical waveguide interface equipment. Optical couplers, cabling, adhesives, or attachments can be included to optically mate laser or detector elements to optical fibers of optical link 430.

Optical connectors 461 and 442 may each include NPC type or PC type connectors. A numerical aperture of a first optical fiber coupled in a connector can be of a different numerical aperture of a second optical fiber coupled to the first optical fiber in the connector. In some configurations, optical connectors 461 and 442 can each include a NPC connector-bulkhead-NPC connector configuration, where an NPC connector is attached to ends of optical fibers to be coupled, and the bulkhead physically couples the two NPC connectors to each other.

FIG. 5 is a system diagram illustrating system 500 for applying optical signals to tissue of a patient. System 500 includes measurement assembly 540, optical cable assembly 560, and tissue interface assembly 510. In operation, optical signals generated by optical source 555 of measurement system 541 are applied to tissue of a patient for measurement of a physiological parameter. Tissue of a patient is not included in this example for clarity, but can be coupled to tissue interface pad 515, as detailed in FIG. 1. Optical signals are transferred via optical link 530 to tissue interface pad 515 for application into the tissue. Optical signals are received through the tissue and transferred to detectors 550-552 of measurement system 541 via optical link 530.

FIG. 5 illustrates portions 570-573 of system 500. These portions are included to highlight different properties of the associated optical fibers of optical link 530. In this example, portion 572 includes optical fibers associated with tissue interface assembly 510, which can be a separate assembly than measurement system 541 and optical cable assembly 560. Tissue interface assembly 510 and the associated optical fibers 511-514 can be detached via optical connector 561 from optical cable 563, and likewise optical cable 563 can be detached via optical connector 562 from the elements of measurement assembly 540 and associated optical links 523 and 525-527. Thus, tissue interface assembly 510, measurement assembly 540, or optical cable assembly 560 can be more readily replaced, disposed, repaired, or swapped separately from each other.

Optical link 430 includes two optical pathways in this example. A first source pathway is for introduction of source optical signals from measurement system 541 into tissue, and a second measurement pathway is for receipt of optical signals from tissue and into measurement system 541. The source pathway and the measurement pathway share common optical connectors 561-562 and common optical cable 563 in this example. Source optical fiber 511 and measurement optical fibers 512-514 are each terminated at one end within tissue interface pad 510 for coupling optical signals to and from tissue of a patient. Optical fibers 511-514 can be all bundled into a sheath or loom, or adhered together. Likewise, optical fibers 523 and 525-527 can be all bundled into a sheath or loom, or adhered together.

The source pathway includes optical fibers 520-522, optical fiber 523, optical fiber 533, and optical fiber 511. Emitter portions of optical source 555 are optically coupled to first ends of associated ones of optical fibers 520-522, and are each configured to emit an optical signal of an associated wavelength. The three different optical signals in this example include respective carrier wavelengths of 670 nm, 795 nm, and 850 nm, although other wavelengths can be used. The optical coupling between elements of optical source 555 and first ends of associated ones of optical fibers 520-522 can include optically transmissive adhesive, optical connectors, or other optical coupling elements. Second ends of optical fibers 520-522 are coupled to a first end of optical fiber 523. In this example, second ends of all three of optical fibers 520-522 couple without connectors or other coupling equipment to the first end of optical fiber 523 via a butt coupling or butt joint. Ends of each fiber 520-523 can be butt joined as shown in detailed view 501, such as by using an optically transmissive adhesive to mate the associated ends of the optical fibers. Thus, while each of optical fibers 520-522 carries an individual optical signal of a single carrier wavelength, these three individual optical signals are merged into optical fiber 523. Optical fiber 523 then carries all optical signals originally carried by optical fibers 520-522. A rigid strain relief can be placed surrounding the butt joint. The rigid strain relief can be a cylinder made of plastic or metal and glued to fibers very near the joint, such as exemplified by strain relief 343 in FIG. 3, although other configurations can be employed. Optical fiber 523 passes through grommet 542 in a casing of measurement system 541 and terminates at optical connector 562. Optical connector 562 optically couples a second end of optical fiber 523 to a first end of optical fiber 533 which is bundled in optical cable 563 with other optical fibers. In alternate configurations, grommet 542 is omitted and connector 562 is coupled to the casing of measurement system 541 and passes the associated optical signals through the casing of measurement system 541. A second end of optical fiber 533 terminates in optical connector 561. Optical connector 561 optically couples the second end of optical fiber 533 to a first end of optical fiber 511. Finally, a second end of optical fiber 511 is terminated by tissue interface pad 510 for introduction of optical signals originally generated by optical source 555 into tissue. A detailed view 502 is shown for optical cable 563 indicating exemplary positions for the associated optical fibers 533 and 535-537. Sheathing, loom, or adhesive material 539 surrounds, bundles, and protects the internal optical fibers of optical cable 563. Optical cable 563 can be a co-extruded fiber assembly with a rugged jacket on the outside. In some examples, the individual optical fibers of optical cable 563 are coated or jacketed in an optically absorbing medium or substance, such as polyamide or opaque adhesive, to prevent optical crosstalk between individual optical fibers. Optical cable 563 can be a co-extruded fiber assembly with a rugged jacket on individual fibers which comprises an absorbing medium. Optical cable 563 can also include a rugged jacket enclosing the entire cable.

The measurement pathway for measurement or output optical signals includes optical fibers 512-514, optical fibers 535-537, and optical fibers 525-527. First ends of optical fibers 512-514 are terminated by tissue interface pad 510 for receipt of optical signals from tissue. First ends of optical fibers 512-514 can be terminated at different locations within tissue interface pad 510, such as shown in FIG. 5 at different distances from the end of source optical fiber 511. Optical fibers 512-514 each receive all optical signals originally emitted into tissue, which can include all three optical signals originally generated by optical source 555 and introduced into the tissue. Second ends of optical fibers 512-514 are terminated in optical connector 561. Optical connector 561 optically couples the second ends of optical fibers 512-514 to first ends of optical fiber 535-537 which are bundled in optical cable 563 with optical fiber 533. Second ends of optical fibers 535-537 terminate in optical connector 562. Optical connector 562 optically couples the second ends of optical fibers 535-537 to first ends of optical fibers 525-527. Optical fibers 525-527 pass through grommet 542 in the casing of measurement system 541, and second ends of optical fibers 525-527 are then coupled to optical detectors 550-552. Thus, optical fibers 535 and 525 carry optical signals originally received by optical fiber 512, optical fibers 536 and 526 carry optical signals originally received by optical fiber 513, and optical fibers 537 and 527 carry optical signals originally received by optical fiber 514.

Also, portions 570-573 can include optical fibers of different optical or physical properties, as well as lengths. The optical or physical properties can include optical attenuation properties, minimum bend radii, optical fiber materials or compositions, numerical apertures, or cross-sectional thicknesses, including variations or combinations thereof. Portion 570 indicates a first optical fiber length associated with optical cable assembly 560, portion 571 indicates a second optical fiber length associated with optical fibers 511-514 of tissue interface assembly 510, portion 572 indicates a third optical fiber length of the ‘pigtail’ portion of measurement assembly 540 composed of optical fibers 523 and 525-527, and portion 573 indicates a fourth optical fiber length of the three source optical fibers 520-522. Although other lengths can be used, typical lengths are 2 meters for portion 570, 0.5 meters for portion 571, 0.5 meters for portion 572, and 0.1 meters for portion 473. It should be noted the optical links of FIG. 5 are not necessarily drawn to scale.

In this example, optical fibers 511-514 are composed of first optical fiber material with a first minimum bend radius and a first optical attenuation per unit length. Optical fibers 533 and 535-537 are each composed of a second optical fiber material with a second minimum bend radius and a second optical attenuation per unit length. Optical fibers 533 and 535-537 each comprise a higher-quality or less attenuating type of optical fiber, such as optical fibers composed of glass. Optical fibers 511-514 each comprise a lower-quality or higher attenuating type of optical fiber, such as optical fibers composed of PMMA. Thus, optical fibers 533 and 535-537 are less attenuating to optical signals per unit length than optical fibers 511-514. Optical fibers 511-514 and 533 and 535-537 are each 1000 micrometers thick. Optical fibers 525-527 can be composed of similar fiber material and thickness as optical fibers 533 and 535-537, although variations are possible. Optical fibers 520-522 can be of any optical fiber material, but in this example are of a smaller thickness than optical fiber 523. Thus, due to the smaller thicknesses, optical fibers 520-522 can all be mated simultaneously to an end of the larger thickness optical fiber 523 as shown in detailed view 501. Optical fibers 520-522 are each 200 micrometers thick, and optical fiber 523 is 1000 micrometers thick, although other thicknesses can be employed.

Returning to the elements of system 500, tissue interface pad 515 may be composed of plastic, foam, rubber, glass, metal, adhesive, or some other material, including combinations thereof. Tissue interface pad 515 includes a generally planar surface configured to interface with tissue to allow for introduction of optical signals into tissue and for receipt of optical signals from tissue. Tissue interface pad 515 also may include elements as discussed above for tissue interface pad 110, 310, or 410, although these elements can use different configurations.

Measurement system 541 includes processing system 545, optical source 555, and optical detectors 550-552. Optical source 555 can include solid state lasers, LEDs, vertical-cavity surface-emitting laser (VCSEL), or other optical sources, including combinations thereof. In this example, optical sources are discrete optical sources, in separate component packages, and couple to associated individual ones of optical fibers 520-522. Alternatively, optical source 555 can instead couple to a single optical fiber such as shown in FIG. 4 for optical source 455 and optical fiber 423. Optical detectors 550-552 each comprise light detector elements, such as a photodiode, phototransistor, avalanche photodiode (APD), photomultiplier tube, charge coupled device (CCD), or other optoelectronic sensor, along with associated receiver circuitry such as amplifiers or filters. Optical detectors 550-552 receives optical signals over optical fibers 525-527, and converts the optical signals into corresponding electrical signals. Processing system 545 can include digital processors, computer systems, microprocessors, circuitry, non-transient computer-readable media, user interfaces, or other processing devices or software systems, and may be distributed among multiple processing devices. Processing system 545 can also include photon density wave (PDW) generation and measurement equipment, electrical to optical conversion circuitry and equipment, optical modulation equipment, and optical waveguide interface equipment. Optical couplers, adhesives, cabling, or attachments can be included to optically mate laser or detector elements to optical fibers of optical link 530.

Optical connectors 561-562 may each include NPC type or PC type connectors. A numerical aperture of a first optical fiber coupled in a connector can be of a different numerical aperture of a second optical fiber coupled to the first optical fiber in the connector. Optical connectors 561-562 are shown as multi-fiber connectors, such as Lightray MPX connectors, although other multi-fiber connectors can be employed. In other configurations, optical connectors 361-362 can each include a NPC connector-bulkhead-NPC connector configuration, where an NPC connector is attached to ends of optical fibers to be coupled, and the bulkhead physically couples the two NPC connectors to each other. In further examples, connector 562 comprises a bulkhead connector coupled to a casing of measurement system 541, and acts to pass the optical signals through the casing of measurement system 541 without the need for grommet 542.

The included descriptions and figures depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the invention. Those skilled in the art will also appreciate that the features described above can be combined in various ways to form multiple embodiments. As a result, the invention is not limited to the specific embodiments described above, but only by the claims and their equivalents.

What is claimed is:

1. A system to optically analyze tissue of a patient, comprising:
a measurement system configured to transfer a plurality of source optical signals over an optical link, receive a plurality of measurement optical signals over the optical link, and process at least the plurality of measurement optical signals to identify a value of a physiological parameter of the patient;
the optical link comprising a first source portion with a first link property configured to carry the plurality of source optical signals and a second source portion with a second link property coupled to the first source portion and configured to carry the plurality of source optical signals; and
a tissue interface assembly configured to receive the plurality of source optical signals transferred over the optical link, emit the plurality of source optical signals into the tissue, and receive the plurality of measurement optical signals from the tissue for transfer over the optical link.

2. The system of claim 1, wherein the first link property and the second link property each comprise at least one of an optical attenuation level, a numerical aperture, a minimum bend radius, and a cross-sectional thickness.

3. The system of claim 1, wherein the first source portion comprises a plurality of source optical fibers, each of the plurality of source optical fibers carrying one of the plurality of source optical signals, and wherein the second source portion comprises an intermediate optical fiber configured to carry all of the plurality of source optical signals.

4. The system of claim 3, wherein first ends of the plurality of source optical fibers are butt coupled to a first end of the intermediate optical fiber.

5. The system of claim 3, wherein the plurality of source optical fibers each comprise a first cross-sectional thickness, and wherein the intermediate optical fiber comprises a second cross-sectional thickness larger than the first cross-sectional thickness.

6. The system of claim 1, the optical link further comprising a third source portion with a third link property coupled to the second source portion and configured to carry the plurality of source optical signals to the tissue interface assembly, wherein the tissue interface assembly is configured to receive the source optical signals over the third source portion of the optical link.

7. The system of claim 6, wherein the first source portion comprises a plurality of optical fibers each of a thickness less than 200 micrometers, wherein the second source portion comprises a glass optical fiber of a thickness greater than 200 micrometers, and wherein the third source portion comprises a polymethyl methacrylate (PMMA) optical fiber of a thickness greater than 200 micrometers.

8. The system of claim 1, the optical link further comprising a first measurement portion comprising a plurality of measurement optical fibers coupled to the tissue interface assembly and each configured to receive the plurality of measurement optical signals from the tissue interface assembly, and a second measurement portion coupled to the first measurement portion and configured to carry the plurality of measurement optical signals to a detector portion of the measurement system.

9. The system of claim 8, the optical link further comprising a third source portion with a third link property coupled to the second source portion and configured to carry the plurality of source optical signals to the tissue interface assembly, wherein the tissue interface assembly is configured to receive the source optical signals over the third source portion of the optical link;
wherein the first measurement portion couples to the second measurement portion via an optical connector, and wherein the third source portion couples to the second source portion via the optical connector, wherein the optical connector comprises a multi-fiber connector.

10. The system of claim 9, wherein at least the second source portion and the second measurement portion are bundled into a single optical cable assembly with a first end of the single optical cable assembly comprising the optical connector.

11. A method of operating a system to optically analyze tissue of a patient, the method comprising:
in a measurement system, transferring a plurality of source optical signals over an optical link, receiving a plurality of measurement optical signals over the optical link, and processing at least the plurality of measurement optical signals to identify a value of a physiological parameter of the patient;
in a first source portion of the optical link, carrying the plurality of source optical signals using a first link property;
in a second source portion of the optical link coupled to the first source portion, carrying the plurality of source optical signals using a second link property; and
in a tissue interface assembly, receiving the plurality of source optical signals transferred over the optical link, emitting the plurality of source optical signals into the tissue, and receiving the plurality of measurement optical signals from the tissue for transfer over the optical link.

12. The method of claim 11, wherein the first link property and the second link property each comprise at least one of an optical attenuation level, a numerical aperture, a minimum bend radius, and a cross-sectional thickness.

13. The method of claim 11, wherein the first source portion comprises a plurality of source optical fibers, each of the plurality of source optical fibers carrying one of the plurality of source optical signals, and wherein the second source portion comprises an intermediate optical fiber configured to carry all of the plurality of source optical signals.

14. The method of claim 13, wherein first ends of the plurality of source optical fibers are butt coupled to a first end of the intermediate optical fiber.

15. The method of claim 13, wherein the plurality of source optical fibers each comprise a first cross-sectional thickness, and wherein the intermediate optical fiber comprises a second cross-sectional thickness larger than the first cross-sectional thickness.

16. The method of claim 11, further comprising:
in a third source portion of the optical link coupled to the second source portion, carrying the plurality of source optical signals to the tissue interface assembly using a third link property; and
in the tissue interface assembly, receiving the source optical signals over the third source portion of the optical link.

17. The method of claim 16, wherein the first source portion comprises a plurality of optical fibers each of a thickness less than 200 micrometers, wherein the second source portion comprises a glass optical fiber of a thickness greater than 200 micrometers, and wherein the third source portion comprises a polymethyl methacrylate (PMMA) optical fiber of a thickness greater than 200 micrometers.

18. The method of claim 11, further comprising:
in a first measurement portion of the optical link comprising a plurality of measurement optical fibers coupled to the tissue interface assembly, receiving the plurality of measurement optical signals from the tissue interface assembly; and
in a second measurement portion of the optical link coupled to the first measurement portion, carrying the plurality of measurement optical signals to a detector portion of the measurement system.

19. The method of claim 18, further comprising:
in a third source portion of the optical link coupled to the second source portion, carrying the plurality of source optical signals to the tissue interface assembly using a third link property;
in the tissue interface assembly, receiving the source optical signals over the third source portion of the optical link;
in an optical connector comprising a multi-fiber connector, coupling the first measurement portion to the second measurement portion, and coupling the third source portion to the second source portion.

20. A system to optically analyze tissue of a patient, comprising:
a measurement system configured to transfer a plurality of source optical signals over an optical link, receive a plurality of measurement optical signals over the optical link, and process at least the plurality of measurement optical signals to identify a value of a physiological parameter of the patient;
the optical link comprising a plurality of source optical fibers each coupled at first ends to associated optical sources of the measurement system that generate individual ones of the plurality of source optical signals, and the plurality of source optical fibers further coupled to an intermediate optical fiber at second ends, wherein the intermediate optical fiber is configured to carry the plurality of source optical signals for delivery to a tissue interface assembly;
the optical link further comprising a plurality of measurement optical fibers configured to receive the plurality of measurement optical signals transferred by the tissue interface assembly at third ends, and each of the plurality of measurement optical fibers further coupled to associated optical detectors of the measurement system at fourth ends; and
the tissue interface assembly comprising a tissue interface pad configured to interface with the tissue to emit the plurality of source optical signals into the tissue and receive the plurality of measurement optical signals from the tissue, and the tissue interface assembly further comprising a tissue interface optical link coupled to the intermediate optical fiber and to the plurality of measurement optical fibers.
